(19) United States
(12) Patent Application Publication
(10) Pub. No.: US 2016/0203729 A1
(43) Pub. Date: Jul. 14, 2016

(54) DYNAMIC INTERACTION SYSTEM AND METHOD

(71) Applicant: Happify, Inc., New York, NY (US)

(72) Inventor: Ran Zilca, Raanana (IL)

(21) Appl. No.: 14/990,380

(22) Filed: Jan. 7, 2016

Related U.S. Application Data
(60) Provisional application No. 62/101,315, filed on Jan. 8, 2015.

(51) Int. Cl. G09B 11/00; A61B 5/16; G09B 5/06

(52) U.S. Cl. CPC G09B 11/00; G09B 5/06; A61B 5/165

(57) ABSTRACT

A method and system are provided for dynamic user interaction. The method includes assessing input data, received from a user, that is used to determine a psychological state of the user. The method further includes determining a current psychological state of the user, using an application stored in non-transitory storage media, based on the input data, and determining possible courses of action for the user based on the determined current psychological state of the user. The method additionally includes presenting the current psychological state and the possible courses of action to the user through a physical interface.

### RELATED APPLICATION INFORMATION

This application claims priority to provisional application Ser. No. 62/101,315, filed on Jan. 8, 2015, incorporated herein by reference in its entirety.

### BACKGROUND

1. Technical Field

The present invention relates to a human interactive system, and more particularly to a method and system for interacting with a user using an application on an electronic device.

2. Description of the Related Art

Well-being, behavior-change, and positive psychology software applications are generally comprised of a set of activities that are arranged together to form an individual program or application. The way in which the activities are arranged and administered to users constitutes an interaction model of an application, sometimes referred to as the “scaffolding” or “structure” of the application. The practical role of the interaction model is to narrow down the possible activities that are offered to a user at any point in time to select from.

Applications that interact with users employ an interaction model. Existing applications use either a random-access/direct/tree interaction model, a school/course interaction model, or a hybrid between these models. All programs currently available on the market use a static model of interaction, where the initiative for interaction is either controlled by the user or by a system. None of the programs use an interaction model that has a notion of a session. The users simply start and stop the activities as they wish.

### SUMMARY

According to an aspect of the present principles, a method is provided for dynamic user interaction. The method includes assessing input data, received from a user, that is used to determine a psychological state of the user. The method further includes determining a current psychological state of the user, using an application stored in non-transitory storage media, based on the input data, and determining possible courses of action for the user based on the determined current psychological state of the user. The method additionally includes presenting the current psychological state and the possible courses of action to the user through a physical interface.

According to another aspect of the present principles, a system is provided for dynamic user interaction. The system includes a user interface, coupled to a hardware processor, configured to receive input data from a user, and a memory configured to store the received input data. The system further includes a dynamic interaction module, coupled to the memory, configured to assess the input data, determine a current psychological state of the user from the assessing of the input data, and determine possible courses of action for the user based on the determined current psychological state of the user. The system additionally includes a physical interface configured to present the current psychological state and the possible courses of action to the user.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram of a system for dynamic interaction with a user, in accordance with an embodiment of the present principles;

FIG. 2 is a diagram showing a conversational interaction model in accordance with the present principles;

FIG. 3 is a state diagram showing assessment results for a user's current psychological state, in accordance with the present principles;

FIG. 4 is a block/flow diagram of a method by which a user may use the application, in accordance with the present principles;

FIG. 5 is a view of the application while in use by a user, in accordance with the present principles; and

FIG. 6 is a flowchart of method for dynamic user interaction, in accordance with the present principles.

It should be understood that the drawings are for purposes of illustrating the concepts of the invention and are not necessarily the only possible configuration for illustrating the invention. To facilitate understanding, identical reference numerals have been used, where possible, to designate identical elements that are common to the figures.

### DETAILED DESCRIPTION OF PREFERRED EMBODIMENTS

In accordance with the present principles, an application using a dynamic interaction module is provided wherein the application performs the function of a positive psychology coach, providing a user with questions or tasks, assessing the user's current psychological state from the responses to the questions or tasks, and supplying the user with suggested courses of action.

The design of the interaction model, in addition to the practicality of limiting user choices, has a dramatic effect on user-engagement. From a theoretical standpoint, the number of choices that are presented to a user at any screen/turn needs to be optimized; not enough choices means reduced perception of autonomy and reduced intrinsic motivation. Too many choices leads to a reduced sense of autonomy as well, and reduced satisfaction: the “paradox of choice.” A design requirement for the interaction model of applications is therefore to reduce the number of choices presented to the user at each time so that they are relevant, in context, and make the choice simple, enticing, and engaging. Most of the programs on the market today fail to meet this goal. As a result, most applications suffer from a very low level of engagement, especially when with sustained usage, e.g., over weeks and months.

Designing applications with engaging interaction models affect the value of the entire market of mobile/digital health and behavior-change. A better interaction model uses self-directed, behavior-change software applications.

Use of any of the following: “at least on of.” “/,” and “and/or,” for example, in the cases of “at least one of X and Y,” “X/Y,” and “X and/or Y” is intended to encompass the selection of the first listed option (X) only, or the selection of the second listed option (Y) only, or the selection of both options (X and Y). As a further example, in the cases of “X, Y, and/or Z” and “at least one of X, Y, and Z”, such phrasing is intended to encompass the selection of the first listed option (X) only, or the selection of the second listed option (Y) only, or the selection of the third listed option (Z) only, or the selection of the first and the second listed options (X and Y) only, or the selection of the first and third listed options (X and Z) only, or the selection of the second and third listed options (Y and Z) only, or the selection of all three options (X and Y and Z). This may be extended, as readily apparent by one of ordinary skill in this and related arts, for as many items listed.

Appearances of the phrase “in one embodiment” or “in an embodiment”, or any other variations of this phrase, appearing in various places throughout the specification are not necessarily all referring to the same embodiment. In the specification, references to “an embodiment” or “one embodiment” of the present principles, as well as variations other than these, mean that a particular characteristic, feature, structure, and so forth described in connection with the embodiment described is included in at least one embodiment of the present principles.

The present principles may be incorporated in a system, a method, and/or the product of a computer program, the product including a computer readable storage medium having program instructions that are readable by a computer, causing aspects of the present invention to be carried out by a processor.

The program instructions are readable by a computer and can be downloaded to a computing/processing device or devices from a computer readable storage medium or to an external computer or external storage device via a network, which can comprise a local or wide area network, a wireless network, or the Internet. Additionally, the network may comprise wireless transmission, routers, firewalls, switches, copper transmission cables, optical transmission fibers, edge servers, and/or gateway computers. Within the respective computing/processing device, a network adapter card or network interface in each computing/processing device receives computer readable program instructions from the network and forwards the computer readable program instructions for storage in a computer readable storage medium.

As herein used, a computer readable storage medium is not to be construed as being transitory signals per se, such as radio waves or other freely propagating electromagnetic waves, electromagnetic waves propagating through a waveguide or other transmission media, or electrical signals transmitted through a wire. The computer readable storage medium may be, but is not limited to, e.g., a magnetic storage device, an electronic storage device, an optical storage device, a semiconductor storage device, an electromagnetic storage device, or any suitable combination of the foregoing, and can be a tangible device that can retain and store instructions for use by an instruction execution device. The following is a list of more specific examples of the computer readable storage medium, but is not exhaustive: punch-cards, raised structures in a groove, or other mechanically encoded device having instructions recorded thereon, an erasable programmable read-only memory, a static random access memory, a portable compact disc read-only memory, a digital versatile disk, a portable computer diskette, a hard disk, a random access memory, a read-only memory, a memory stick, a floppy disk, and any suitable combination of the foregoing.

The operations of the present invention may be carried out by program instructions which may be machine instructions, machine dependent instructions, microcode, assembler instructions, instruction-set-architecture instructions, firmware instructions, state-setting data, or either source code or object code written in any combination of one or more programming languages, including an object oriented programming language such as, but not limited to, C++, and other conventional procedural programming languages. The program instructions, while having the capability of being executed entirely on the computer of the user, may also be executed partly on the computer of the user, partly on a remote computer and partly on the computer of the user, entirely on the remote computer or server, or as a stand-alone software package. In the “entirely on the remote computer or server” scenario, the remote computer may be connected to the user's computer through any type of network, including a wide area network or a local area network, or the connection may be made to an external computer. In some embodiments, electronic circuitry including, e.g., field-programmable gate arrays, programmable logic circuitry, or programmable logic arrays may execute the program instructions by utilizing state information of the program instructions to personalize the electronic circuitry, in order to perform aspects of the present invention.

These program instructions may be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. These program instructions may also be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The computer readable program instructions may also be loaded onto a computer, other programming apparatus, or other device to produce a computer implemented process, such that the instructions which execute on the computer, other programmable apparatus, or other device implement the functions/acts specified in the flowchart and/or block diagram block or blocks.

Aspects of the present invention are described herein with reference to block and/or other diagrams and/or flowchart illustrations of methods, apparatus, and computer program products according to the present invention's embodiments. It will be understood that each block of the block and/or other diagrams and/or flowchart illustrations, and combinations of blocks in the block and/or other diagrams and/or flowchart illustrations, can be implemented by program instructions that are readable by a computer.

The block and/or other diagrams and/or flowchart illustrations in the Figures are illustrative of the functionality, architecture, and operation of possible implementations of systems, methods, and computer program products according to the present invention's various embodiments. In this regard, each block in the block and/or other diagrams and/or flowchart illustrations may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently or sometimes in reverse order, depending upon the functionality involved. It will also be noted that each block of the block and/or other diagram and/or flowchart illustration, and combinations of blocks in the block and/or other diagram and/or flowchart illustration, can be implemented by special purpose hardware-based systems that perform the specified functions or acts or carry out combinations of special purpose hardware and computer instructions.

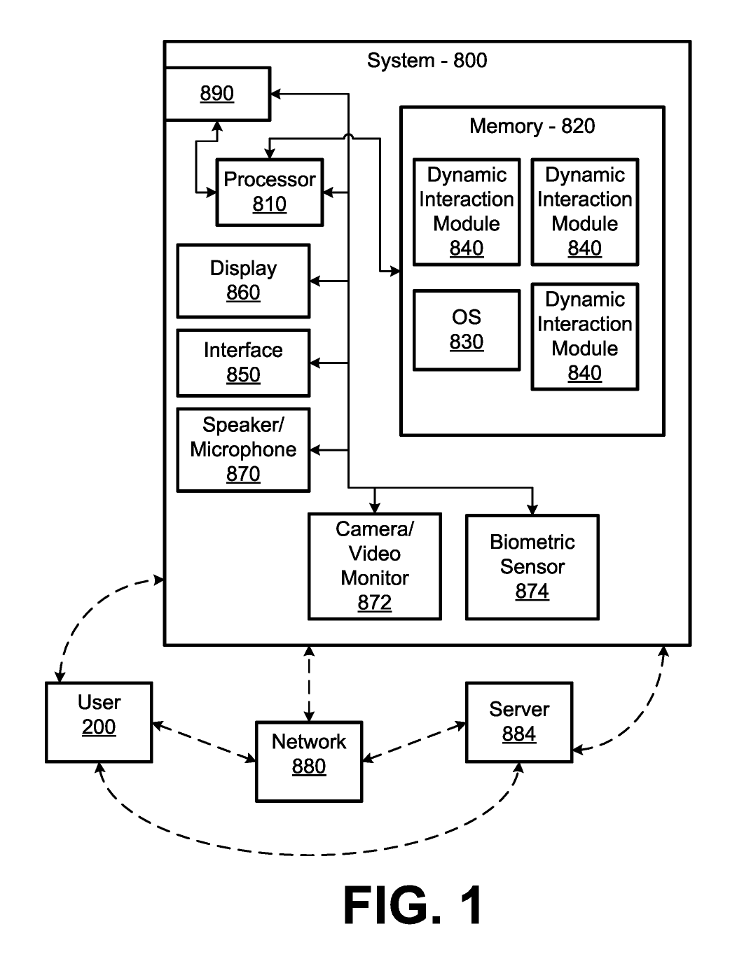

Referring now to the drawings in which like numerals represent the same or similar elements and initially to FIG. 1, a system 800 for dynamic interaction with a user 200 is illustratively shown in accordance with one embodiment. System 800 includes one or more dynamic interaction modules 840 to provide a positive psychology coach and permits natural language interaction between a user or users 200 and the system 800. The dynamic interaction modules 840 analyze the visual, acoustic, biometric, etc. input of the user 200 and, based on this analysis, determine an appropriate method of responding to the user. These methods of responding to the user 200 may include, e.g., synthetic speech, a visual avatar, typed or printed words, etc.

The system 800 is configured to provide the user 200 with inspiration, questions, tasks, etc. which are employed to assess the user's current psychological state from user 200 responses and other data input from the user 200, such as, e.g., blood pressure, heart rate, facial inflections, speech, etc. The responses may include gestures, speech, text, facial expressions, eye movements, heart rate, sweat or any other physiological, verbal, acoustic or visual feedback. The user 200 is then supplied with suggested courses of action, and the system 800 tracks the progress and holds the user accountable for his/her progress. The courses of action may be, e.g., interventions or, more particularly, Digital Behavior Change Interventions (DBCIs).

The system 800 may include a computer device configured to interact with the user 200. The computer device may include a mobile device (e.g., smartphone, tablet, etc.) or even a specially designed computer device.

The system 800 preferably includes one or more processors 810 and memory 820 for storing programs and applications. Memory 820 may store an operating system 830 and other programs and applications. Memory 820 stores a dynamic interaction module 840 configured to provide the functionality as described herein in accordance with the present principles.

In one embodiment, the system 800 includes a display 860 (which may include an interactive touch screen display) for interacting with the system 800. Display 860 permits the user to interact with the components and functions, or any other element within the system 800. This is further facilitated by an interface 850 which may include a keyboard, mouse, a joystick, a haptic device, or any other peripheral or control to permit user feedback from and interaction with the system 800. Interface 850 may include a camera, gesture recognition device, microphone/speaker (also separately depicted as microphone/speaker 870). System 800 may further include an I/O port 890 for inputting and outputting data.

Additionally, the system 800 may further include at least one speaker/microphone 870, camera/video monitor 872, or biometric sensor 874. These components 870, 872, 874 may send signals to the interface 850 and may include a heart rate viability sensor, a galvanic skin response sensor, an activity sensor, a breathing sensor, an electrocardiography sensor, an electroencephalography sensor, a sleep sensor, etc. Of course, other types of sensors may also be employed, while maintaining the spirit of the present principles. In one embodiment, the sensors 870, 872, 874 are incorporated into a mobile device, such as a smart phone. In another embodiment, the sensors 870, 872, 874 are separate electronic devices.

The system 800 may be part of or otherwise be connected to a network 880 and coupled to a server or service provider 884. The network 880 may include wireless communications, wired communication, etc. The network 880 may include the Internet, a wide area or local area network, etc. The server 884 may be one computer acting as a server. The server 884 may also be a plurality of electronic or server devices acting as a virtual server. The broken lines in FIG. 1 signify that the user 200, network 880, server 884, and system 800 may be connected to any one or more of the user 200, network 880, server 884, and system 800, either directly, indirectly, or remotely over a communication path. One or more of the system 800, network 880, and server 884 may be located on one computer, distributed over multiple computers, or be partly or wholly Internet-based.

The interaction models 840 may be a design component of Interactive Voice Response Systems (IVR), since interfacing may be done over the phone (with no visuals), and the number of possible choices provided to a user may be narrowed down to a minimum. Calling an IVR may result in going through endless menus to listen to possible options, only to realize that none of them fit the reason for the call. Unlike ineffective interaction models, the present model does not result in long wait times until the appropriate activity is heard, or in a situation where none of the offered activities are appropriate. The present model creates relatively short wait times and optimizes the number and content of the offered activities.

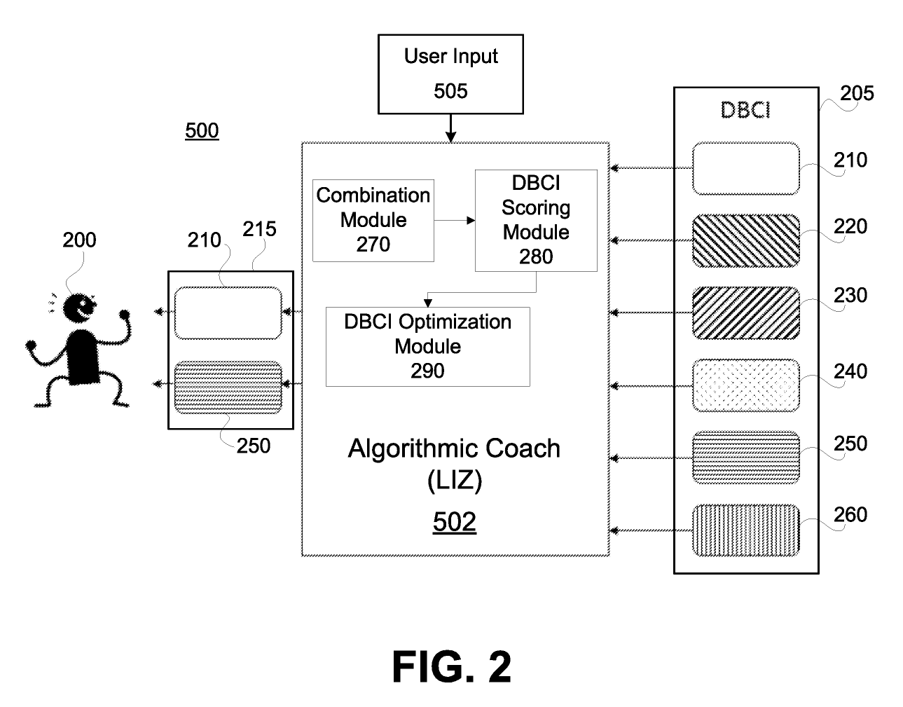

Referring now to FIG. 2, a diagram of a conversational interaction model 500 is shown in accordance with the present principles. In the conversational interaction model 500, a list 205 of DBCIs 210, 220, 230, 240, 250, 260 are available to an algorithmic coach 502. In one embodiment, the algorithm coach 502 has a name. The name of the algorithm coach 502 may be, e.g., an uncommon female name that is not associated with any ethnicity (e.g., “Liz”).

DBCIs are interactive, automated packages of advice and ongoing support for behavior change, which may include: personalized advice based on responses to questions assessing needs, circumstances and preferences; support for goal-setting, planning and progress monitoring; automated reminders and progress-relevant feedback and encouragement; access to social support by email, online forums, etc. DBCIs can be employed for a wide range of different behaviors; for example, to reduce risky or antisocial behavior, increase productivity in the workplace, enhance learning activities, or support environmentally important lifestyle change, such as reducing energy use. DBCIs are a method of supporting behavior change and provide personalized interactive support. DBCIs provide a way of carrying out detailed assessments of the process of behavior change from a much larger sample of the population than has previously been possible. DBCIs may be delivered by PCs and provide feedback to users based on their answers to questions about their activities and feelings.

The algorithmic coach 502 selects only those DBCIs 510, 520, from a set of DBCIs 504, which are most relevant to the user. These DBCIs 210, 220 are based upon user-provided information and/or feedback 505. This limits the number of DBCIs 215 from which the user 200 can choose. The user-provided information may include, e.g., verbal information, sensor-acquired data, video, blood pressure, heart rate, breathing, and facial expressions. Of course, other types of user-provided information 505 may also be employed, while maintaining the spirit of the present principles.

Once the user-provided information 505 is input into the system 800 (FIG. 1), a combination module 270 combines all of the various types of user-provided information and/or measurements 505. Once the user-provided information and/or measurements 505 is combined, the user-provided information and/or measurements 505 is analyzed by employing a DBCI scoring module 280. Once this analysis is complete, the DBCI scoring module compares the results of the analysis with the list of DBCIs 205 and assesses the relevance of each of the DBCIs 210, 220, 230, 240, 250, 260 to the user-provided information and/or measurements 505. This is performed by providing a score to each of the DBCIs 210, 220, 230, 240, 250, 260 based on the results of the analysis. Based on the application's default settings and/or the user-provided information and/or measurements 505, a single DBCI may be selected or a combination of DBCIs may be selected. There may also be a feedback loop by which the DBCI scoring module 280 repeatedly analyzes the incoming user-provided information and/or measurements 505 and compares the user-provided information and/or measurements 505 to the list of DBCIs 205. Once the DBCIs are scored, a DBCI optimization module 290 is used to determine which of the DBCIs 210, 220, 230, 240, 250, 260 are most relevant to the user 200.

As an example of this process, if the user-provided information 505 is speech, the DBCI scoring module 280 may include a Natural Language Understanding (NLU) module in order to convert the speech to text and determine possible emotional or psychological attributes within the speech (e.g., sadness or happiness). In this example, once these possible psychological or emotional attributes are determined, the DBCI scoring module 280 would provide a score to the individual DBCIs 210, 220, 230, 240, 250, 260 based on the prevalence of those attributes within those particular DBCIs 210, 220, 230, 240, 250, 260. After this scoring is complete, the DBCI optimization module 290 would determine which of the DBCIs in this list of DBCIs 205 are the most relevant to the user based on the scores given to each of the DBCIs 210, 220, 230, 240, 250, 260 by the DBCI scoring module 280.

In another embodiment, the algorithmic coach 502 determines which DBCIs 210, 220 to make available to the user 200 based on the user's 200 assessed psychological state. The user's psychological state is not necessarily a fixed value and may change over time.

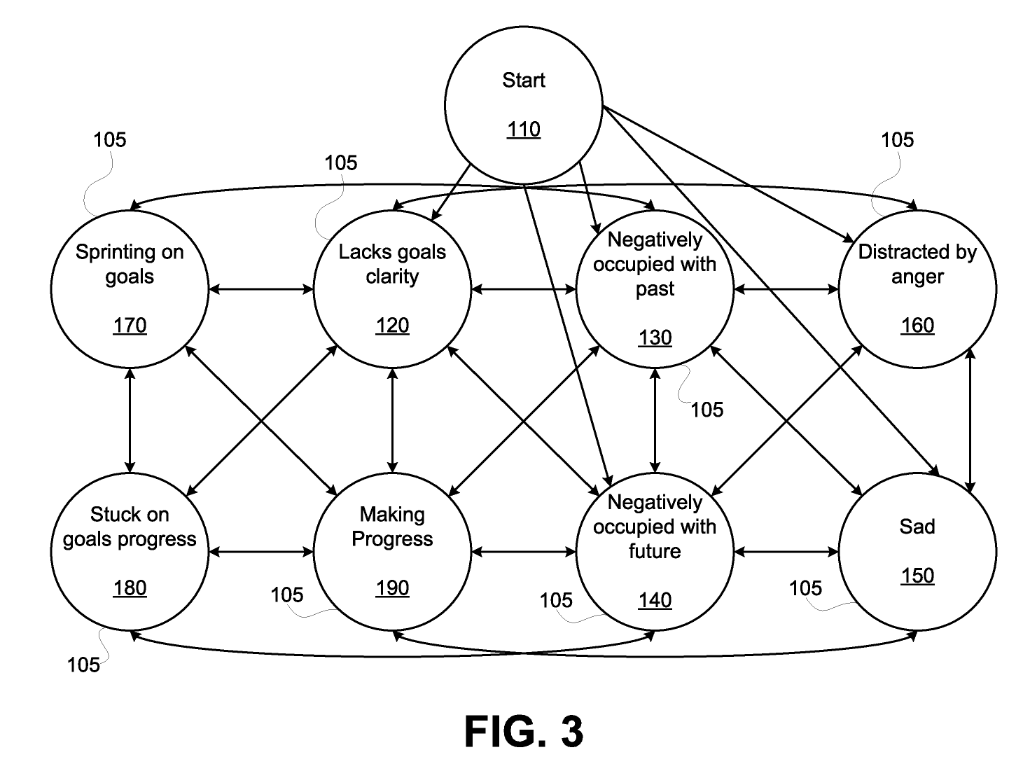

Referring now to FIG. 3, a state diagram showing assessment results 105 for a user's current psychological state is shown, in accordance with one embodiment of the present principles. A user starts an application 110. After an assessment of the user's current psychological state 105 is conducted, the user's current psychological state 105 is determined. This determination is accomplished by analyzing the responses that the user input into the system, which may include gestures, speech, text, facial expressions, eye movements, heart rate, sweat or any other physiological, verbal, acoustic or visual feedback.

Following the initial assessment of the user's psychological state 105, further assessments are later conducted. These assessments can determine if the user's prior psychological state has changed to one of the other psychological states. As the arrows indicate, the change in the user's psychological state 105 has many possibilities. The assessments are determined based on conditional changes on user inputs (direct and indirect). These changes may include, e.g., an increase/decrease in blood pressure or heart rate, a change in the pitch of the user's speech, a change in the user's facial expressions, a manual update by the user of the user's mood, etc. In one embodiment, the input data may comprise data collected by applying analytic techniques to the user input data while interacting with DBCIs.

The current psychological state and the possible courses of action may be displayed to the user on a display, such as the display 860 described in FIG. 1. The system may be programmed to interact with the user on the display using a visual avatar. The avatar may be expressive and affective and have the capability of conveying emotional expressions.

In one embodiment, the current psychological state and the possible courses of action may be transmitted to the user through a speaker. The system may be programmed to interact with the user through the speaker using a voice interface. The voice interface may be expressive and affective and have the capability of conveying emotional audible expressions.

In another embodiment, the current psychological state may be automatically extracted from the DBCIs, e.g., in the form of text.

The initial psychological states 105 may include “Lacks goals clarity” 120, “Negatively occupied with past” 130, “Negatively occupied with future” 140, “Sad” 150, and “Distracted by anger” 160. This list of psychological states is non-exhaustive and other psychological states are contemplated. Each of these psychological states 105 correlates to different mental attitudes, feelings, outlooks, etc. The user may be assessed as “Lacks goals clarity” 120 if the assessment has determined, from the user input, that the user is uncertain about what goals the user would like to accomplish. The user may be assessed as “Negatively occupied with past” 130 if the assessment has determined that the user, rather than focusing on ultimate goals, is focused on past occurrences in a negative light. The user may be assessed as “Negatively occupied with future” 140 is the assessment has determined that the user, rather than having a positive outlook on the goals that he/she can accomplish, is focused on the negative events that can occur in the future. The user may be assessed as “Sad” 150 if the assessment has determined that the user has feelings of unhappiness or sorrow. The user may be assessed as “Distracted by anger” 160 if the assessment has determined that the user is aggravated or has feelings of hostility and this aggravation or hostility is distracting the user.

The psychological states may change after the first assessment and may include, e.g., “Lacks goals clarity” 120, “Negatively occupied with past” 130, “Negatively occupied with future” 140, “Sad” 150, “Distracted by anger” 160, “Sprinting on goals” 170, “Stuck on goals progress” 180, and “Making progress” 190. This list of psychological states is non-exhaustive and other psychological states are contemplated. The user may be assessed as “Sprinting on goals” 170 if the assessment has determined that the user is rapidly approaching accomplishing his/her goals. The user may be assessed as “Stuck on goals progress” 180 if the assessment has determined that the user is not making any significant progress toward accomplishing his/her goals. The user may be assessed as “Making progress” 190 if the assessment has determined that the user is gradually making progress towards accomplishing his/her goals.

In one embodiment of the present principles, the psychological states may represent the user's readiness to change, state of change, emotional state, actionable state, the state of the user's progress towards goals, or a number of DBCIs completed by the user. Identification of the goals can be done by a statistical classifier, using various machine learning methods, e.g., Hidden Markov Model, decision tree, artificial neural network, genetic algorithm, Bayesian classifier, etc. The statistical classifier is trained using reference data to identify the psychological state of the user out of a finite number of possible states. In addition to direct text user input, the statistical classifier can use physiological sensors (e.g., galvanic skin response, Electromyography (EMG) brain waves, heart rate, heart rate variability, breath), mobile data (e.g., Global Positioning System (GPS) location, accelerometer and speed, orientation of the device in space, pedometer), and social data from other people (e.g., interaction with friends and family, calls, texts, activity on social networks). In another embodiment, the interface is performed using acoustic feedback, such as speech. The system 800 (FIG. 1) can extract psychological cues from the speech signal, e.g., emotional state or self-determination/motivation. The system 800 (FIG. 1) may also use text analysis methods, e.g., n-gram language models.

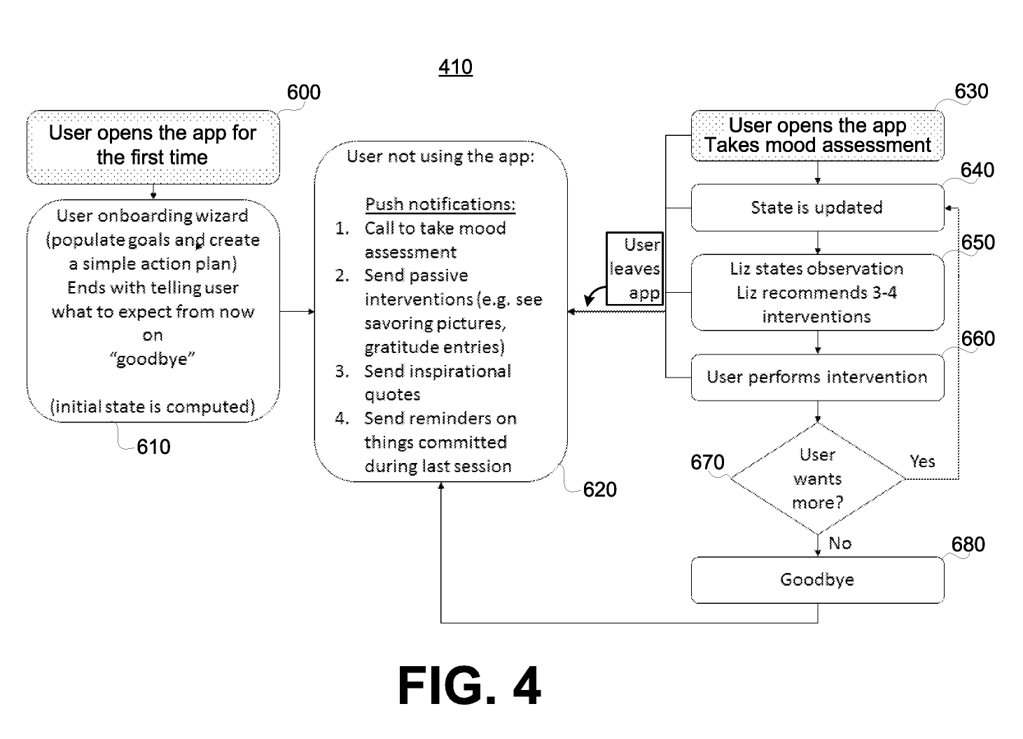

Referring now to FIG. 4, a block/flow diagram of a method 410 by which a user 200 may use the application is shown in accordance with the present principles. The method 410 can begin at block 600 at which point the user opens the application for the first time. At this point, no assessment has yet been conducted on the user's current psychological state.

The user 200 employs the program for the first time and, at block 610, enables an onboarding wizard. The onboarding wizard guides the user 200 to populate their account with goals and to create a simple action plan. In one embodiment, when the user 200 is finished inputting the desired information, the application sends a message to the user 200, explaining what the user is to expect from the application from this point forward. If the user is aware of what steps are to come, there is likely to be less confusion on the part of the user. In another embodiment, the application ends the message to the user with “goodbye.”

If the user has stopped using the application, the application performs block 620. At block 620, when the user is not using the application, the application will send push notifications to the user. In one embodiment, the push notifications may include calling the user to take a mood assessment. In another embodiment, the push notifications may include sending the user passive interventions, such as sending the user savoring pictures or gratitude entries. In another embodiment the push notifications may include sending the user inspirational quotes. In yet another embodiment, the push notifications may include sending the user reminders on things that the user committed to during a last session with the application.

One of the benefits of the application is that it guides the user through a session 410. During the session 410, a user opens the application 630, the user's psychological state is updated 640 (if this is not the user's first session), the algorithm coach 502 (FIG. 2) states observations and recommends interventions 650, the user performs at least one of the interventions 660, the user indicates 670 whether (s)he wants the algorithm coach to restart the process at block 640, and the session ends 680.

At block 630, the user 200 begins a session with the application. The user 200 opens the application and takes a mood assessment. After taking the mood assessment, the user's current psychological state is determined.

In one embodiment, the mood assessment includes a direct questionnaire in which the user scores a set of statements to a degree of their validity, and then an algorithm computes a composite score from the scores of the individual statements. The user may also attach a wearable or other sensor that measures psychologically-indicative physiological characteristics such as galvanic skin response, blood pressure, and heart rate variability, and the results taken from the sensor are used to determine the current psychological state of the user.

In another embodiment, an algorithm is used that analyzes all of the user's activity, including direct input from questionnaires and sensors and the history of actions taken, their content, their frequency, and their trend. The algorithm uses all of this data to determine the current psychological state of the user

At block 640, the user's 200 psychological state is updated to conform with the assessed current psychological state. In one embodiment, after the psychological state is updated, a dynamic interaction module 840 (FIG. 1) sends a greeting to the user.

The dynamic interaction module 840 sends a message to the user, stating observations the dynamic interaction module 840 may have. In one embodiment, the observations may include a description of how much time has passed since the user's last session. In another embodiment, the observations may include a congratulatory message for any goals that the user has completed.

The dynamic interaction module 840 (FIG. 1) may decide on a small set of DBCIs 525 (FIG. 2) to present to the user. These DBCIs 525 are interventions and the dynamic interaction module 840 recommends, e.g., 3 to 4 interventions with suggestions on what actions the user may take. The interventions may be determined based on the assessed current psychological state of the user. In an embodiment, the model 500 (FIG. 2) would be a Finite State Machine (“FSM”). The FSM may be programmed initially by an expert based on past research, and in the future be trained using statistical methods like Hidden Markov Models. The FSM may analyze recent trends in the user's mood and then use these trends to assess the user's current mood and determine appropriate interventions.

The individual activities used in previous wellness applications lose much of their value because the activities are not provided in context, e.g., a user could open the application and be directed or choose to think about the user's “best possible self.” In contrast, in the present embodiment of a coaching/therapy session, a user, based on the results of the current session or of previous sessions, may reach the conclusion that (s)he is overly worried about the future and has no clarity about his/her future goals and, as a result, should consider to engage in such an activity. This conclusion is thus not based solely on a direct request of the user to think of his/her “best possible self,” but rather is a process by which the session guides the user to come to the conclusion.

In another embodiment, if the user is assessed to be sad, the dynamic interaction module 840 (FIG. 1) may present interventions that are mood-boosting. If the user is assessed to be negatively occupied with the future, the dynamic interaction module 840 may present optimistic interventions. The suggested interventions may also be submitted by other users. In this example, the user may send a question to the online ether and gets notified if and when another user responds with an answer.

At each assessed psychological state, a machine learning algorithm may be utilized to synthesize an optimal action to take, according to the state classification. The system 800 (FIG. 1) may decide to elicit more input from the user or alternatively to respond to the user. In one embodiment, the system 800 may adapt to the individual user by altering the wording of a response. In yet another embodiment, the system 800 may use speech output and a speech signal may be programmed with emotional cues, e.g., emphasizing certain words or giving the words certain emotional color.

At block 660, the user performs the intervention or interventions that the dynamic interaction module has suggested in block 650. If the user ceases using the application during blocks 630, 640, 650, or 660, the application returns to block 620. If the user continues to use the application after block 660, block 670 is performed.

At block 670, the user is asked whether more feedback from the dynamic interaction module is desired. If the user indicates that more feedback is desired, the application returns to block 640. If the user indicates that no more feedback is desired at this time, the application proceeds to block 680. At block 680, the application sends a farewell message to the user and returns to block 620.

The application should be designed as a sequence of “turns”—just like any other conversational/dialog application. This means that, whether it's a push notification or a single turn within a session, the application is an ongoing sequence of turns. Therefore, the application's main interface will be designed similarly to an old “terminal interface,” where all turns show, and the user is engaged with the most recent turn. The simplicity of this design could make it very easily applicable to web in addition to mobile.

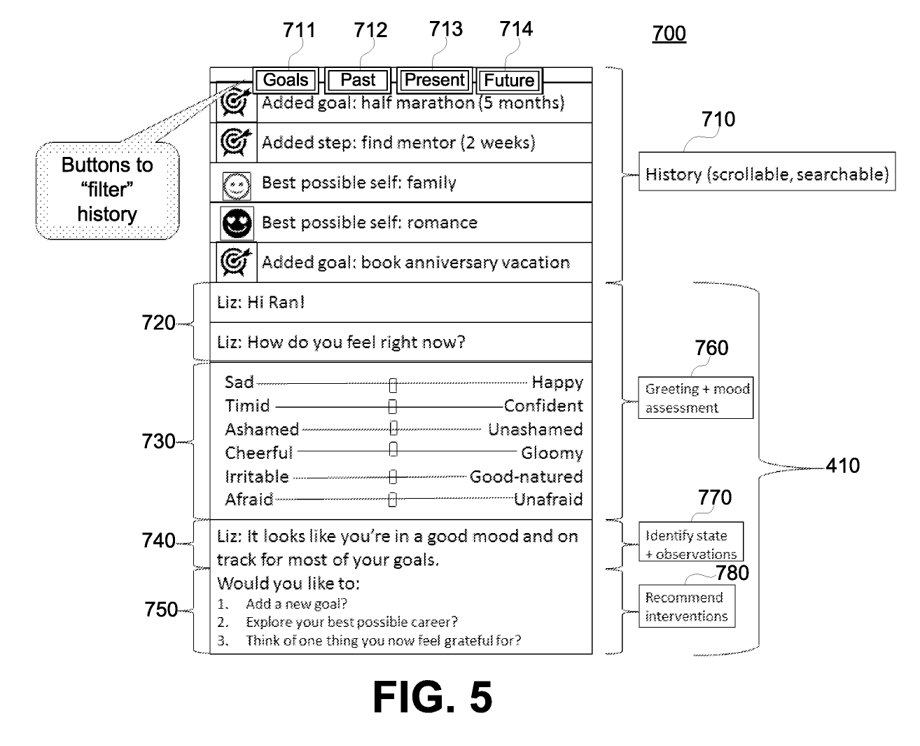

Referring now to FIG. 5, a view of the application while in use by a user 200 is shown in accordance with the present principles. After the user 200 has performed at least one session 410 with the dynamic interaction model, some of the user's goals will be in the system as well as at least one of the user's psychological states and the results of any “best possible self” exercises. These goals may include, e.g., “half marathon (5 months), “find mentor (2 weeks),” “book anniversary vacation,” etc., and the results of the “best possible self” exercises may include, e.g., “family,” “romance,” etc. When the user 200 is using the application 700, the user 200 will have access to the user's history 710, greetings 720, mood assessment 730, last assessed psychological state and observations 740, and recommended interventions 750.

In one embodiment, the user 200 can filter the user's history. In another embodiment, the history is filtered by selecting buttons 711, 712, 713, 714 that correlate to different aspects of the user's 200 history 710. These buttons may include user's goals 711, past 712, present 713, and future 714. In one embodiment, clicking on “past” 712 will show “replay happy day.” In another embodiment, clicking on “present” 713 will show savoring album photos. In yet another embodiment, clicking on “future” 714 will show all results of “best possible self” exercises.

The greetings 720 may include messages from the dynamic interaction module 840 to the user 200. These messages may include, e.g., “Liz: Hi Ran!,” or “Liz: How do you feel right now?” The messages may be standard messages from the dynamic interaction module 840 or may be specific to the user 200.

The mood assessment 760 includes a sliding scale 730 which allows the user 200 to rank their mood between two opposing mood categories. The opposing mood categories may be “Sad” and “Happy,” “Timid” and “Confident,” “Ashamed” and “Unashamed,” “Cheerful” and “Gloomy,” “Irritable” and “Good-natured,” and/or “Afraid” and “Unafraid.” Of course, other mood categories may also be employed, while maintaining the spirit of the present principles.

After the mood assessment, the psychological state is identified 770. In one embodiment, the dynamic interaction model's observations from previous sessions and an explanation of the user's current assessed psychological state 740 are presented to the user 200. This may include, e.g., “Liz: It looks like you're in a good mood and on track for most of your goals.” Interventions are then recommended 780 and the dynamic interaction module 840 may send messages 750 to the user 200 which may include, e.g., activities that the dynamic interaction module has determined are appropriate actions to take following the current assessment of the user's psychological state. This message 750 may be, e.g., “Would you like to: 1. Add a new goal? 2. Explore your best possible career? 3. Think of one thing you now feel grateful for?”

The user 200 may start a new behavior, stop a current behavior, increase a behavior, etc. in the course of the user's 200 overall interaction with the application. In this embodiment, the dynamic interaction module 840 uses these factors in its determination of the interventions.

The dynamic interaction module 840 may include a system to increase the user's medication adherence where the user's 200 goals are medication adherence.

In one embodiment of the present principles, the dynamic interaction module 840 includes a system to help users 200 manage chronic medical conditions, e.g., diabetes.

In another embodiment of the present principles, the dynamic interaction module 840 includes a system that helps users 200 manage weight and/or attain preventative/promotional health goals.

In yet another embodiment of the present principles, the dynamic interaction module 840 includes a system to help users 200 determine their optimal career path. The system may also employ Efficient Second-order Minimization (ESM) to do tracking and monitoring.

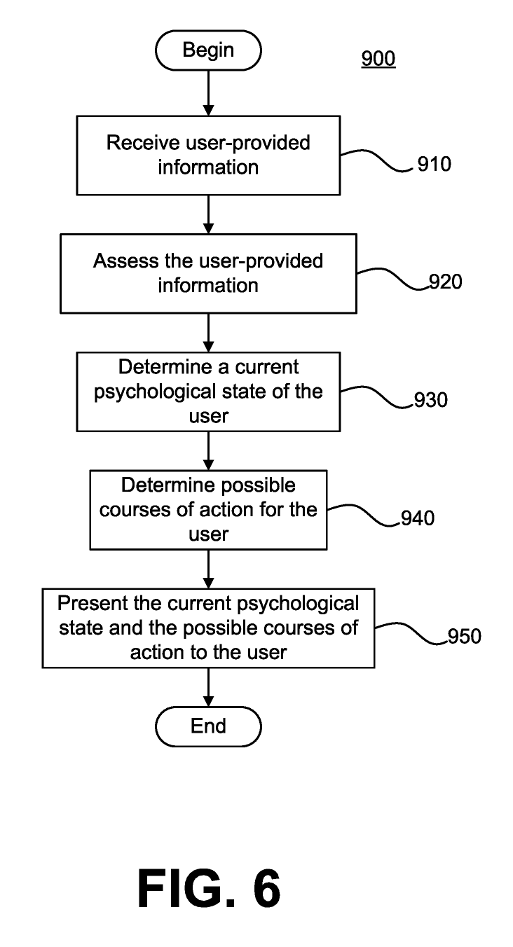

Referring now to FIG. 6, a flowchart of method 900 for dynamic user interaction is shown, in accordance with one embodiment of the present principles.

At block 910, user-provided information 505 is sent to, and received by, the system 800. The user-provided information 505 may be relevant to the user's 200 psychological state and may include, e.g., verbal information, sensor-acquired data, video, etc.

At block 920, the user-provided information is assessed by the system 800. This assessment process 920 may include combining multiple types of user-provided information 505 and comparing the data 505 to the data of known psychological states. Once the user-provided information 505 is assessed, the current psychological state of the user is determined, at block 930, using the results of the assessment 920 of the user-provided information 505.

At block 940, once the current psychological state of the user 200 is determined, the system 800 determines possible courses of action for the user to take. This determining process 940 may be performed by comparing the user-provided information to a set of DBCIs 205.

At block 950, once the possible courses of action are determined, the current psychological state and the possible courses of action are presented to the user. This may be performed using a physical interface such as, e.g., a display or a speaker. The method of presenting the current psychological state and the possible courses of action may include, e.g., video, sound, typed or printed text, etc.

Having described preferred embodiments of a system and method of a dynamic interaction system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A method for dynamic user interaction, comprising:
assessing input data, received from a user, that is used to determine a psychological state of the user;
determining a current psychological state of the user, using an application stored in non-transitory storage media, based on the input data;
determining possible courses of action for the user based on the determined current psychological state of the user; and
presenting the current psychological state and the possible courses of action to the user through a physical interface.

2. The method according to claim 1, wherein the current psychological state can change from one psychological state to a different psychological state.

3. The method according to claim 3, wherein the current psychological state is automatically extracted from Digital Behavior Change Interventions.

4. The method according to claim 1, wherein the physical interface includes a display.

5. The method according to claim 4, further comprising interacting with the user on the display using a visual avatar, wherein the visual avatar conveys synthesized emotions.

6. The method according to claim 1, wherein the physical interface includes a speaker.

7. The method according to claim 6, further comprising interacting with the user through the speaker using a voice interface, wherein the voice interface conveys synthesized emotions.

8. The method according to claim 1, wherein the input data comprises user answers to a questionnaire.

9. The method according to claim 1, wherein the input data comprises data collected from sensors.

10. The method according to claim 9, wherein the sensors comprise at least one biometric sensor.

11. The method according to claim 1, wherein the input data comprises data collected by applying analytic techniques to the user input data while interacting with Digital Behavior Change Interventions.

12. A system for dynamic user interaction, comprising:
a user interface, coupled to a hardware processor, configured to receive input data from a user;
a memory configured to store the received input data;
a dynamic interaction module, coupled to the memory, configured to:
assess the input data;
determine a current psychological state of the user from the assessing of the input data; and
determine possible courses of action for the user based on the determined current psychological state of the user; and
a physical interface configured to present the current psychological state and the possible courses of action to the user.

13. The system according to claim 12, wherein the current psychological state can change from one psychological state to a different psychological state.

14. The system according to claim 12, wherein the dynamic interaction module is further configured to automatically extract the current psychological state from Digital Behavior Change Interventions.

15. The system according to claim 12, wherein the physical interface includes a display.

16. The system according to claim 15, wherein the dynamic interaction module is further configured to interact with the user on the display using a visual avatar, wherein the visual avatar conveys synthesized emotions.

17. The system according to claim 12, wherein the physical interface includes a speaker.

18. The system according to claim 17, wherein the dynamic interaction module is further configured to interact with the user through the speaker using a voice interface, wherein the voice interface conveys synthesized emotions.

19. The system according to claim 12, further comprising sensors configured to send the input data to the user interface.

20. The system according to claim 19, wherein the sensors comprise at least one biometric sensor.
